(19) United States
(12) Patent Application Publication
(10) Pub. No.: US 2011/0177115 A1
(43) Pub. Date: Jul. 21, 2011

(54) VACCINATION REGIMEN

(76) Inventors: Tomas Hanke, (US); Gerald Wayne Both, North Ryde (AU)

(21) Appl. No.: 12/897,533

(22) Filed: Oct. 4, 2010

(30) Foreign Application Priority Data
Oct. 2, 2009 (AU) 2009904813

(51) Int. Cl. A61K 39/12; A61K 39/295; A61P 37/04

(52) U.S. Cl. 424/199.1

(57) ABSTRACT

The present invention provides a method of raising an immune response in a subject against an antigen. The method involves the steps of a) administering to the subject a non-atadenoviral vector comprising a nucleic acid encoding the antigen, and b) administering an engineered atadenovirus to the subject, wherein the genome of the engineered atadenovirus encodes the antigen. Step b) is performed after step a).

### FIELD

The invention relates to a method of raising an immune response in a subject against an antigen. In particular, the invention relates to a method of vaccinating a subject against an antigen comprising the use of an atadenovirus in the vaccination regime.

### BACKGROUND

There are many infectious diseases such as HIV-1, hepatitis C and malaria where disease prevention would be superior to treatment, which is not completely effective. As millions of people around the world are susceptible to these and other infectious diseases the cost of treating individuals with drugs would be prohibitive. However, if effective vaccines could be developed the risk of acquiring disease would be greatly reduced by mass vaccination programmes that have proved to be effective for diseases such as smallpox and poliomyelitis. Similarly, new forms of cancer treatment involving vaccination may be developed using antigens expressed by the cancer cells so that the immune system can eradicate the cancer.

Vaccination involves an attempt to stimulate an immune response in a subject so that it is able to combat the infectious agent if it is encountered or recognize tumour antigens if they are expressed. Vaccination may stimulate the subject to produce antibodies that bind to and inactivate the infectious agent or activate key cell types of the immune system. In particular, CD4+ and CD8+ T cells are key components of an effective immune response to eliminate cancer or infected cells and to clear them from the body.

Vaccines may be produced in several ways. For example, they may contain the crude, inactivated infectious agent, or they may be derived by attenuation of the infectious agent which is administered as a live vaccine. Alternatively, protein antigens may be purified from the agent and administered in an appropriate formulation. However, antigen purification may be tedious and is not always applicable. More recently, genes that encode antigens have been incorporated into vectors and delivered into the body where they are expressed. For antibody-inducing vaccines, the genes may code for proteins whose shapes mimic the native antigens as closely as possible. For T cell inducing vaccines, the gene may encode whole or chimaeric proteins or linked immunogenic epitopes (polyepitope proteins). After gene delivery, immunogens expressed in the cell are processed into peptides and presented to the immune system by MHC class I complexes, which stimulates CD8+ cytotoxic T cells. In some cases expressed antigens may be found outside the cell, in which case they are taken up by professional antigen presenting cells, processed and presented by MHC class II complexes, which stimulates CD4+ T helper cells. These may then recruit other immune cells locally or help CD8+ T cells and antibody production.

Numerous types of gene delivery vectors for vaccination have been described. These include vectors based on plasmid DNA and several types of viruses. In particular, vectors based on poxviruses such as Modified Vaccinia virus Ankara (MVA) and human adenoviruses (HAdV) may be mentioned as these have been widely used for vaccination. A vector derived from ovine atadenovirus (OAdV) has also been developed. This virus is the prototype of the genus atadenovirus. Other members of the genus are found in ruminants (cattle, deer and goats), possum, ducks and reptiles.

The intention of vaccination is to induce an immune response to the expressed passenger antigen. However, attenuated non-replicating vectors are not likely to be sufficiently immunogenic to induce protective immune responses following a single vaccine administration. In addition, the vector is recognized as a foreign agent and a response is also directed against vector components. As a consequence, gene delivery by a second and subsequent dose of vector is increasingly reduced. To overcome this problem prime/boost protocols have been developed in which a different vector is used to deliver the same antigen on each subsequent occasion. Examples are provided in patent applications such as WO 9739771, U.S. Pat. No. 7,273,605 and WO 2004037294. In WO 9739771 methods of inducing a CD8+ T cell response against an antigen are provided which use combinations of poxvirus and adenovirus vectors expressing the same antigen. U.S. Pat. No. 7,273,605 similarly uses combinations of non-replicating pox virus vectors to induce an immune response to an antigen. WO 2004037294 uses HAdV vectors from rarer human serotypes in combination with each other or with HAdV5. However, in most cases a preferred order of vector administration that may induce an enhanced immune response is not specified. One exception to this is EP 1335023 which specifies a kit containing a priming composition to induce a CD8+ immune response. The priming agent may be chosen from DNA, a virus-like particle or a non-replicating AdV but the boosting agent should be MVA or a strain derived from it. Another exception is EP 1214416 which specifies the use of a non-replicating AdV vector for boosting a CD8+ T cell immune response primed by a composition comprising the antigen or epitope or nucleic acid encoding the antigen or epitope delivered as DNA, a virus-like particle or MVA. These examples indicate that the preferred order of vector administration to induce an optimal cellular immune response cannot be predicted and that the preferred order may change depending on the combination of vectors used.

Because its receptors are unidentified and its interaction with the immune system is not fully understood, OAdV vectors expressing particular antigens were used in the present study with combinations of other vectors expressing the same antigen to determine whether there is an optimal order of administration. Surprisingly, it was found that there is a preferred order of vector administration that induces a higher level of antigen-specific T cell immunity than any other vector. This discovery could not have been predicted based on the known properties and history of the individual vectors but it has utility in the development of future vaccination strategies.

### SUMMARY

Accordingly, the present invention provides a method of raising an immune response in a subject against an antigen, comprising the steps of a) administering to the subject a non-atadenoviral vector comprising a nucleic acid encoding the antigen, and b) administering an engineered atadenovirus to the subject, wherein the genome of the engineered atadenovirus encodes the antigen, wherein step a) is performed before step b). In one embodiment, the atadenovirus is OAdV.

Step b) of the method may be performed 1 day to 10 years, preferably 1 to 12 weeks, after step a). The antigen in step a) may be administered using a range of non-atadenoviral vectors such as plasmid pTH, poxvirus (eg. MVA), or mastadenoviruses such asHAdV, ChAdV, PAdV and/or BAdV.

In one embodiment, step a) is repeated at least once prior to step b). Typically, the antigen administered in the repeated step a) is not administered by an engineered OAdV.

In one embodiment the method results in an enhanced T cell response. The enhanced T cell response may be an enhanced CD8+ and/or CD4+ T cell response.

### BRIEF DESCRIPTION OF THE DRAWINGS

FIG. 1. Induction of HIV-1-specific T cell responses by HAdV5 and OAdV vectors expressing HIVA as measured by peptides H and P (grey and black bars, respectively).

FIG. 2. Induction of HIV-1-specific T cell responses by DNA, MVA and OAdV vectors expressing HIVA. (A, B) CD8+ T cells as measured by peptides H and P (grey and black bars, respectively). (C) CD4+ T cell responses.

### DETAILED DESCRIPTION

It is to be understood that the present invention is not limited to particularly exemplified methods, analysis, subjects, diseases or conditions which may vary. It is also to be understood that the terminology used herein is for the purpose of describing particular embodiments of the present invention only, and is not intended to be limiting.

All publications, patents and patent applications cited herein, whether above or below, are hereby incorporated by reference in their entirety. However, publications mentioned herein are cited for the purpose of describing and disclosing the protocols and reagents which are reported in the publications and which might be used in connection with the subject invention. Nothing herein is to be construed as an admission that the instant invention is not entitled to antedate such disclosure by virtue of prior invention.

Furthermore, the practice of the present invention employs, unless otherwise indicated, conventional molecular biology, pharmaceutical and immunological techniques within the skill of the art. Such techniques are well known to the skilled worker, and are explained fully in the literature.

As used in the subject specification, the singular forms “a”, “an” and “the” include plural aspects unless the context clearly dictates otherwise. Thus, for example, reference to an “antigen” includes a single antigen as well as two or more antigens, “a subject” includes a single subject or two or more subjects. Reference to “the invention” includes single or multiple aspects of the invention.

Throughout the specification the word “comprise” and variations of the word, such as “comprising” and “comprises”, means “including but not limited to” and is not intended to exclude other additives, components, integers or steps. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Unless defined otherwise, all technical and scientific terms used herein have the same meanings as commonly understood by one of ordinary skill in the art to which this invention belongs. Although any materials and methods similar or equivalent to those described herein can be used to practice or test the present invention, the preferred materials and methods are now described.

The present invention is based on the surprising elucidation that when OAdV is used as a vector to boost an immune response to an antigen, significantly higher immunity can be induced than when the vector order is changed. Without wishing to be bound by theory, the present inventors believe this may be because non-ovine subjects will not have been exposed to OAdV prior to the vaccination regimen and therefore will not have developed antibodies which neutralise the OAdV. Accordingly, the OAdV containing a nucleic acid which encodes the antigen of interest will be able to infect the cells of the non-ovine subject. For this reason, the inventors believe that any atadenovirus is suitable for use in the present invention, provided it is not administered to the animal species which it naturally infects. The atadenovirus comprising a nucleic acid encoding the antigen may express the antigen, or may merely deliver the nucleic acid molecule encoding the antigen to a cell of a subject, which cell subsequently expresses the antigen.

OAdV is a member of the Adenoviridae family. This is a large family of non-enveloped, icosahedral, generally nonpathogenic viruses with a double-stranded DNA genome. The family Adenoviridae comprises four genera (Mastadenovirus, Aviadenovirus, Siadenovirus, and Atadenovirus). Of these, the best studied are the mastadenoviruses, which include viruses from numerous mammalian species and all known human AdVs. In contrast, members of the avi-, si-, and atadenoviruses have been isolated from birds, although atadenoviruses also occur in mammalian and reptilian species. The prototype Atadenovirus, an ovine isolate (serotype 7; OAdV), is the only member for which any significant biological studies have been undertaken. OAdV uses a different (unknown) receptor from human adenovirus AdV5 and infects, but does not replicate in, human cells. No antibodies to OAdV have so far been found in human sera and OAdV is not neutralised by sera that neutralise HAdV5 vectors. OAdV will only replicate in certain ovine cell lines. It is able to infect non-ovine cells, including many human cell types but replication is abortive even though OAdV vectors retain a full gene complement. OAdV replication in human cells remains abortive even in the presence of a replicating human AdV. Infection occurs via a primary receptor that is unidentified but distinct from CAR, the major HAdV5 receptor. It is not known whether OAdV uses a secondary receptor such as integrin for infection, but no obvious integrin binding motifs have been identified within OAdV capsid proteins. The interactions between OAdV and immune cells remain to be elucidated but by binding to certain cell surface proteins OAdV vectors may modulate different cell signalling pathways and thus induce an immune response with different characteristics compared to other AdV. OAdV has a genome structure that is significantly different from AdV in other genera but many features are conserved within the genus. There are unique structural genes and numerous non-structural genes whose functions are unknown but which no doubt contribute to the overall biological properties. Recombinant OAdV vectors have been used to deliver genes for reporter proteins and antigens to mice. A single dose of vector can stimulate an antigen-specific immune response. In vivo, OAdV vectors are broadly distributed to major organs in the body such as the heart, spleen and kidney, reflecting the distribution of the receptor(s) used. Unlike many HAdV, OAdV does not accumulate in the liver probably due to the lack of interaction between its hexon protein and certain blood clotting factors. Collectively, the above properties suggest that OAdV is a safe vector which has significant potential for vaccination. Further information regarding OAdV can be found in U.S. Pat. Nos. 7,037,712, 7,091,030 and 6,020,172, the disclosures of which are incorporated herein by reference.

Accordingly, in one embodiment the invention provides a method of vaccinating a subject against an antigen. The antigen may be any antigen encoded by a nucleic acid molecule since atadenovirus is used as a vector for delivering the antigen-encoding nucleic acid to a cell of a subject. Unless stated otherwise, the term antigen includes both the proteinaceous form of the antigen as well as a nucleic acid encoding the proteinaceous form. In one embodiment, the antigen is a membrane protein, an intracellular protein or an extracellular protein since all of these proteins would be exposed to the immune system of the subject to be vaccinated either directly or via the antigen presenting machinery of the cell. Specific examples of suitable proteins include enzymes, structural proteins and binding proteins.

Terms such as “antigen”, “immunogen”, “antigenic fragment” or the like mean a molecule that comprises one or more epitopes that are capable of stimulating a subject's immune system to make, e.g., a secretory, humoral or cellular antigen-specific response against the antigen, immunogen or fragment. The antigen may be a complete protein or a fragment of a protein (peptide), or a nucleic acid encoding either of these. Antigenic fragments are synthetic or natural derivatives of natural or intact antigens or immunogens that retain at least a detectable capacity, e.g., at least about 10%, 20%, 30%, 40%, 50% or more of the native antigen's antigenic capacity, to stimulate a subject's immune system in a desired manner. The antigen may be derived from nature or synthesised chemically.

As will be understood, the method of the present invention is a “prime boost” method. In the present invention, the antigen is prime administered as a vector comprising a nucleic acid encoding the antigen wherein the vector is not an atadenovirus. For example, the nucleic acid encoding the antigen may be prime administered using a poxvirus or human adenovirus. Poxvirus vectors include modified vaccinia virus Ankara (MVA), a highly attenuated strain which is incapable of replication in primate cell types and fowl pox and canary pox vectors, which can only replicate in avian cells. Among the adenovirus vectors, HAdV type 5 has been widely used but many individuals acquire immunity to the vector due to natural infection. Therefore, vectors based on other serotypes of HAdV and vectors derived from non-human AdV such as bovine (BAdV), porcine (PAdV), and chimpanzee (ChAdV) AdVs are being investigated for use in humans. All are members of the mastadenovirus genus. In another embodiment the nucleic acid encoding the antigen is initially administered in combination with a plasmid, such as pVAX1, pCDNA.3 or pVAC1/2, which are commercially available from Invitrogen or Invivogen or pTH (Hanke, T. et al, 2000), pORT 1 (Cranenburgh, R. M. et al, 2001), V1Jn 1 (Montgomery, D. L. et al, 1997) or other plasmids of similar design.

“Immunization” means the process of inducing a detectable and continuing moderate or high level of antibody or cellular immune response that is directed against an antigen to which the subject has been exposed. Such responses are typically detectably maintained for at least about 3 months to 10 years, or more.

Following the prime administration of the antigen, the subject may be boost administered with an atadenovirus comprising a nucleic acid molecule encoding the antigen. The term boosting in this respect is meant amplifying an immune response such, that when said animal is exposed to said antigen after the amplification, the immune response to said antigen is increased in magnitude compared to before said amplification. In one embodiment, the boost administration of the antigen is in the form of a nucleic acid encoding the antigen in combination with OAdV.

The time-frame between the prime and boost vaccinations will depend upon the antigen being administered, the route of administration, and characteristics of the subject such as age, weight and sex.

The antigen may be administered to the subject more than twice. For example, a further administration may occur between the prime and boost administrations. Any further administration of the antigen will typically, but not necessarily, use the antigen in a different form from that in the prime or boost administrations. Similarly, any further administration of the antigen may be via a different route from that in the prime or boost administrations. In one embodiment, the antigen is prime administered in combination with HAdV, further administered in combination with OAdV, then boost administered in combination with HAdV. In another embodiment, the antigen is prime administered in combination with pTH, further administered in combination with MVA, then boost administered in combination with OAdV.

In any of the administration steps, the antigen can be administered by one or more suitable routes, e.g., oral, buccal, sublingual, intramuscular (i.m.), subcutaneous (s.c.), intravenous (i.v.), intradermal, another parenteral route or by an aerosol. The method of delivery determines the dose of DNA required to raise an effective immune response.

The dose of vector, for example atadenovirus, will be whatever is required to deliver an effective dose of antigen. An effective dose or an effective amount of antigen is one that is sufficient to result in, e.g., a detectable change in a symptom or an immune parameter such as one described herein. An effective dosage (or daily dosage) may be administered to a subject over a period of time, e.g., at least about 1-14 days before a symptom change or an immune parameter detectably changes. Saline injections require variable amounts of DNA, from 10 μg-1 mg, whereas gene gun deliveries require 100 to 1000 times less DNA than intramuscular saline injection to raise an effective immune response. Generally, 0.2 μg-20 μg of DNA are required, although quantities as low as 16 ng have been reported. The optimal quantity of DNA required will depend on factors such as the subject and the antigen.

The present invention is further described by the following non-limiting Examples.

### EXAMPLES

Both genes used in this study encode polyepitope proteins. HIVA consists of consensus HIV-1 clade A Gag p24 and p17 regions coupled to a string of CD8+ T cell epitopes (Hanke and McMichael, 2000) (WO 01/47955). HTVconsv (Létourneau et al., 2007) (WO 2006/123256) similarly links CD8+ T cell epitopes from various clades of HIV-1 and is designed to induce broader protection after vaccination. In some cases the HIVconsv protein was modified with an N-terminal signal peptide sequence (e.g., from tissue plasminogen activator; tPA) and/or a C-terminal membrane anchor domain (e.g., from LAMP1) to assist its expression and reduce potential cellular toxicity. In other situations the gene was split and expressed in several parts to allow administration to different anatomical sites. The gene sequences are available at Genbank Accession numbers BD349499 (HIVA) and CS669324 (HIVconsv). The genes were introduced into the various vectors as described below using standard techniques in molecular biology well known to those skilled in the art.

The pTH.HIVA plasmid DNA was prepared as described previously (Hanke and McMichael, 2000) and prepared for vaccination using the Endo-Free Gigaprep (Qiagen) and stored at −20° C. until use. Construction of MVA.HIVA and MVA.HIVconsv was described previously (Hanke and McMichael, 2000) (Nkolola et al., 2004). Working vaccine stocks were grown in chicken embryo fibroblast cells using Dulbeco's Modified Eagle's Medium supplemented with 10% FBS, penicillin/streptomycin and glutamine, purified on a 36% sucrose cushion, titred and stored at −80° C. until use.

Recombinant E1-deleted HAdV5 vectors expressing HIVA or HIVconsv were obtained using the AdEasy™ Adenoviral Vector System (Stratagene), following the manufacturer's instructions. The vectors also expressed the green fluorescent protein as a marker. Working virus stocks were grown on HEK 293T cells, purified using column chromatography, titred to determine the number of infectious units (IU) determined as GFP-expressing cells and stored at −80° C. until use.

OAdV vectors were constructed as follows. The HIVA and HIVconsv genes were excised from the respective pTH plasmid DNAs and inserted into appropriate restriction sites between the Rouse sarcoma virus (RSV) promoter and BGH polyadenylation signal in plasmids OAdV shuttleR and OAdVshuttleL which were both derived from pRSVpoly (Loser et al., 2003) by introducing new flanking site sequences containing AscI and RsrII sites. Alternatively, portions of the HIVconsv gene were amplified by PCR using strategies well known in the art. Primers were designed that created restriction enzyme sites for cloning together with a 5′ initiation codon and a 3′ sequence including a termination codon distal to sequences encoding an epitope for monoclonal antibody recognition. Well known epitopes such as c-myc, His6 or V5 could be used. The expression cassettes were excised using AscI and RsrII and inserted in the leftward or rightward orientation into the modified plasmid pOAdVcos3 that carries the full length OAdV genome plus unique RsrII and AscI sites introduced at cloning site III (Löser et al., 2003). Purified ˜41 kbp plasmids were digested with I-SceI to release the linear viral genome and DNA was transfected into permissive CSL503 ovine foetal lung cells for virus rescue (Both et al., 2007). Viruses with the correct restriction site profile were passaged up to four times to ensure that the genome was stable. Virus propagation, titration and purification was performed according to published procedures (Both et al., 2007). Infectious particles (TCID50 units (IU)/ml) and total particle (vp/ml) titres were determined and vectors were stored at −80° C. until use. Gene expression was confirmed by Western blot using a mouse anti-V5-Tag monoclonal antibody (Serotec, Cat No 1360) (which recognizes the C-terminal Pk epitope of HIVA and HIVconsv) or another appropriate antibody, corresponding to the epitope used. Alkaline phosphatase-conjugated anti-mouse IgG (Sigma, Cat. No A-3562) (1 in 1,000 dilution) was used as the detection antibody.

### Vaccination and Preparation of Splenocytes.

Groups of four to six 5- to 6-week-old female BALB/c mice were immunized intramuscularly (i.m.) under general anaesthesia using individual vectors doses as specified in the examples below. An equivalent dose of empty vector was used as a control where appropriate. On the day of sacrifice, spleens were collected and splenocytes were isolated by pressing spleens individually through a cell strainer (Falcon) using a 5-ml syringe rubber plunger. Following the removal of red blood cells with Rbc Lysis Buffer (Sigma), splenocytes were washed and resuspended in RPMI 1640 supplemented with 10% FCS, penicillin/streptomycin.

### Ex Vivo IFN-γ ELISPOT Assay

The ELISPOT assay was performed using the Becton Dickinson IFN-γ ELISPOT kit according to the manufacturer's instructions. The membranes of the ELISPOT plates (BD Immunospot™ ELISPOT Plates) were coated with purified anti-mouse IFN-γ antibody diluted in PBS to a final concentration of 5 μg/ml at 4° C. overnight, washed once in R-10, and blocked for 2 h with R-10. A total of 2.5×10⁵ splenocytes were added to each well, stimulated with or without peptide for 16 h at 37° C., 5% CO2 and lysed by incubating twice with deionized water for 5 min. Wells were then washed 3× with PBS 0.05% Tween-20, incubated for 2 h with a biotinylated anti-IFN-γ antibody diluted in PBS 2% FCS to a final concentration of 2 μg/ml, washed 3× in PBS 0.005% Tween-20 and incubated with 50 mg/ml horseradish peroxidase-conjugated to avidin in PBS 2% FCS. Wells were washed 4× with PBS 0.005% Tween-20 and 2× with PBS before incubating with an AEC substrate solution [3-amino-9-ethyl-carbazole (Sigma) dissolved at 10 mg/ml in Dimethyl formaldehyde and diluted to 0.333 mg/ml in 0.1 M acetate solution (148 ml 0.2 M acetic acid and 352 ml 0.2 M sodium acetate in 1 liter pH 5.0) with 0.005% H₂O₂]. After 5-10 min, the plates were washed with tap water, dried and the resulting spots counted using an ELISPOT reader (Autoimmune Diagnostika GmbH).

### Statistical Analysis

One- or two-way ANOVA was used to test for overall differences using Stata version. Where significant differences existed, contrasts were used to determine significance between experimental groups of interest using Bonferroni's correction for multiple comparisons. When the experimental groups represented time between administration of the treatment and sacrifice, linear regression analysis was used for analysis. The underlying assumptions of ANOVA or linear regression were tested and the data transformed by taking logs, if necessary. Data were presented as mean±SD unless otherwise noted. Differences were considered significant at p<0.05.

### Example 1

Various alternating regimens of HAdV5.HIVA (A) and OAdV.HIVA (O) were assessed. Vaccines were administered at weeks 0, 3 or 6 with termination at week 7 (Table 1).

TABLE 1
OAdV.HIVA dose 10⁷ iu i.m.; HAdV5.HIVA 10⁶ iu i.m.)

| | BALB/c | Week 0 | Week 3 | Week 6 | Week 7 |
|---|---|---|---|---|---|
| 1 | 4 | HAdV5 | OAdV | nil | euthanase. |
| 2 | 4 | OAdV | HAdV5 | nil | euthanase. |
| 3 | 4 | HAdV5 | HAdV5 | HAdV5 | euthanase. |
| 4 | 4 | OAdV | OAdV | OAdV | euthanase. |
| 5 | 4 | HAdV5 | OAdV | HAdV5 | euthanase. |
| 6 | 4 | OAdV | HAdV5 | OAdV | euthanase. |

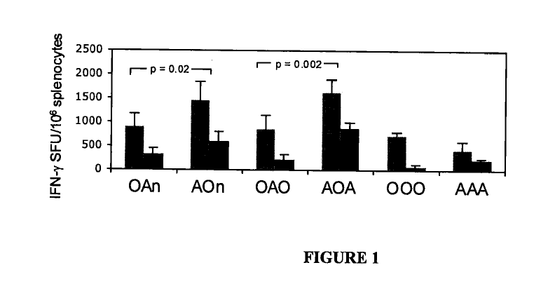

The read out employed the immunodominant epitope H(RGPGRAFVTI; H-2Dd) either alone or in parallel with subdominant epitope P (IFQSSMTKI; H-2 Kd). This series of immunizations demonstrated that as expected heterologous prime/boost regimens were more immunogenic than vaccination with multiple doses of homologous vectors. However, a preferred order of administration emerged. AOn vs OAn just reached significance (p=0.04) and consistent with this, AOA vs OAO was highly significant (p<0.005) (FIG. 1). Therefore, OAdV.HIVA efficiently boosted HAdV5.HIVA primed responses. As the OA versus OAO and AO versus AOA responses were not significantly different (FIG. 1), it is likely that the third vector administration in each case was ineffective due to immunity induced by the first dose of homologous vector. Consistent with this, triple homologous vaccination regimens offered no benefit over a single vaccine delivery (FIG. 1).

### Example 2

OAdV vector immunogenicity was also explored in combination with other vectors using pTH.HIVA DNA (D), MVA.HIVA (M) or the O and A vectors also expressing HIVA (Table 2A).

TABLE 2A
OAdV.HIVA dose 10⁷ iu i.m.; HAdV5.HIVA 10⁶ iu
i.m; MVA.HIVA 10⁶ pfu i.m.; pTH.HIVA DNA (100 ug)

| | BALB/c | Week 0 | Week 3 | Week 6 | Week 7 |
|---|---|---|---|---|---|
| 1 | 4 | DNA | HAdV5 | MVA | euth. |
| DAM | | | | | |
| 2 | 4 | DNA | OAdV | MVA | euth. |
| DOM | | | | | |
| 3 | 4 | DNA | MVA | OAdV | euth. |
| DMO | | | | | |

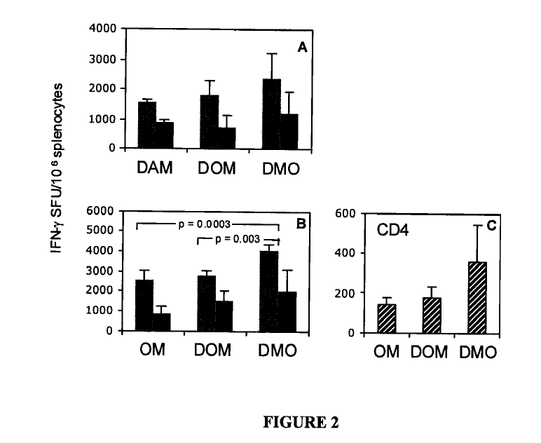

Vector combinations were administered sequentially at weeks 0, 3 and 6 with termination at week 7. DAM, DOM or DMO prime/boost combinations induced high frequencies of H and P peptide-specific T cells (FIG. 2A). Relative immunogenicity could be arranged into an improving hierarchy for H peptide recognition, with DMO the best combination. However, for the P peptide, no combination reached significance compared with another because of the spread in the data points.

A series of high-dose immunizations was also tested. Mice were immunized at weeks 0, 4 and 8 with termination at week 9 (Table 2B) using 100 μg of pTH.HIVA DNA, 10⁹ IU of OAdV.HIVA and 10⁷ PFU of MVA.HIVA.

TABLE 2B
OAdV.HIVA dose 10⁹ iu i.m.; MVA.HIVA
10⁷ pfu i.m.; pTH.HIVA DNA (100 ug)

| | BALB/c | Week 0 | Week 4 | Week 8 | Week 7 |
|---|---|---|---|---|---|
| 1 | 4 | DNA | HAdV5 | MVA | euth. |
| 2 | 4 | DNA | OAdV | MVA | euth. |
| 3 | 4 | DNA | MVA | OAdV | euth. |

The highest mean frequency of splenocytes recognizing the CD8+ T cell epitope H was again achieved by DMO regimen (FIG. 2B). Thus, priming followed by two heterologous booster doses (DMO) was superior to one boost (OM) (p=0.0003) and the order DMO was preferred to DOM (p=0.003). When a mix of three previously identified MHC class II-restricted peptides MHQALSPRTLNAQVKVIEEK, NPPIPVGDIYKRWIILGLNK, and FRDYVDRFFKTLRAEQATQE were used for restimulation the same DMO regimen also induced the highest mean frequency of CD4+ splenocytes (FIG. 2C). Thus, OAdV is the preferred boost vector in these vaccination regimens.

### Example 3

Heterologous prime boost vaccination could be carried out with combinations of chimpanzee (ChAdV; C), OAdV and HAdV5 vectors expressing the HIVconsv antigen as shown in Table 3. ChAdV and OAdV are favoured because they are likely to avoid pre-existing antibodies in human sera that would neutralize HAdV5 vectors. This experiment is designed to confirm the results obtained with HIVA antigen using a second antigen and to demonstrate the preferred pairing and preferred order of vector administration. Because of the lack of cross-reactivity between vectors it would also demonstrate that boosting with the final vector occurs in the face of pre-existing immunity to previously used vectors. The AC and CA pairs are not included on the basis that HAdV5 vectors would not be used in the clinic. Groups ACO and AOC are included for comparison only.

TABLE 3
(OAdV.HIVconsv dose 10⁷ ip i.m.; HAdV5 HIVconsv
10⁶ iu i.m.; ChAdV HIVconsv 10⁶ pfu i.m.)

| | BALB/c | Week 0 | Week 3 | Week 6 | Week 7 |
|---|---|---|---|---|---|
| 1 | 4 | Nil | HAdV5 | OAdV | euth. |
| AO | | | | | |
| 2 | 4 | Nil | ChAdV | OAdV | euth |
| CO | | | | | |
| 3 | 4 | Nil | OAdV | ChAdV | euth. |
| OC | | | | | |
| 4 | 4 | HAdV5 | OAdV | ChAdV | euth. |
| AOC | | | | | |
| 5 | 4 | HAdV5 | ChAdV | OAdV | euth |
| ACO | | | | | |

### Example 4

Heterologous prime boost vaccination using DNA, MVA and ChAdV vectors expressing the HIVconsv gene in combination with OAdV.HIVconsv could be performed as shown in Table 4. DMO and DAO will be included for comparison with earlier studies with HIVA antigen. The experiment will determine the preferred order of vector administration.

TABLE 4
(OAdV.HIVconsv dose 10⁷ ip i.m.; AdHu5 HIVconsv 10⁶ iu i.m.;
MVA HIVconsv 10⁶ pfu i.m.); ChAdV HIVconsv 10⁶ pfu i.m.)

| | BALB/c | Week 0 | Week 3 | Week 6 | Week 7 |
|---|---|---|---|---|---|
| 1 | 4 | DNA | HAdV5 | OAdV | euth |
| DAO | | | | | |
| 2 | 4 | DNA | MVA | OAdV | euth. |
| DMO | | | | | |
| 3 | 4 | DNA | MVA | ChAdV | euth. |
| DMC | | | | | |
| 4 | 4 | DNA | ChAdV | OAdV | euth. |
| DCO | | | | | |
| 5 | 4 | DNA | OAdV | ChAdV | euth. |
| DOC | | | | | |

Similar experiments could be carried out using OAdV vectors that express a sub-portion of the complete HIVconsv gene as this would also demonstrate the ability of OAdV to boost the immune response to the HIVcons antigen expressed by other vectors.

### REFERENCES CITED

Both, G., Cameron, F., Collins, A., Lockett, L., and Shaw, J. (2007). Production and release testing of ovine atadenovirus vectors. Methods Mol. Med. 130, 69-90.
Cranenburgh, R. M., J. A. J. Hanak, S. G. Williams, and D. J. Sherratt. 2001. Escherichia coli strains that allow antibiotic-free plasmid selection and maintenance by repressor titration. Nucleic Acids Research 29:e26.
Hanke, T., and McMichael, A. J. (2000). Design and construction of an experimental HIV-1 vaccine for a year-2000 clinical trial in Kenya. Nat. Med 6 (9), 951-955.
Létourneau, S., Im, E.-J., Mashishi, T., Brereton, C., Bridgeman, A., Yang, H., Dorrell, L., Dong, T., Korber, B., McMichael, A. J., and Hanke, T. (2007). Design and Pre-Clinical Evaluation of a Universal HIV-1 Vaccine. PLoS ONE 2 (10), e984.
Löser, P., Hofmann, C., Both, G. W., Uckert, W., and Hillgenberg, M. (2003). Construction, Rescue, and Characterization of Vectors Derived from Ovine Atadenovirus. J. Virol. 77 (22), 11941-11951.
Montgomery, D. L., J. B. Ulmer, J. J. Donnelly, and M. A. Liu. 1997. DNA Vaccines. Pharmacology & Therapeutics 74:195-205.
Nkolola, J. P., Wee, E.-T., Im, E. J., Jewell, C. P., Chen, N., Xu, X. N., McMichael, A. J., and Hanke, T. (2004). Engineering RENTA, a DNA prime-MVA boost HIV vaccine tailored for Eastern and Central Africa. Gene Ther 11 (13), 1068-1080.

We claim:

1. A method of raising an immune response in a subject against an antigen, comprising the steps of a) administering to the subject a non-atadenoviral vector comprising a nucleic acid encoding the antigen, and b) administering an engineered atadenovirus to the subject, wherein the genome of the engineered atadenovirus encodes the antigen, wherein step a) is performed before step b).

2. The method as claimed in claim 1 wherein the atadenovirus is OAdV.

3. The method as claimed in claim 1 wherein step b) is performed 1 to 12 weeks, after step a).

4. The method as claimed in claim 1 wherein the vector in step a) is selected from the group consisting of pTH, poxvirus (eg. MVA), HAdV, ChAdV, PAdV and BAdV.

5. The method as claimed in claim 1 wherein step a) is repeated at least once prior to step b).

6. The method as claimed in claim 1 wherein the method results in an T cell response.

7. The method as claimed in claim 6 wherein the T cell response is a CD8+ response.

8. The method as claimed in claim 1 wherein the T-cell response is a CD4+ response.
